(19) United States
(12) Patent Application Publication
(10) Pub. No.: US 2016/0303203 A1
(43) Pub. Date: Oct. 20, 2016

(54) COMPOSITIONS AND METHODS FOR THE TREATMENT OF WOUNDS

(71) Applicant: Keshav MALSHE, Orlando, FL (US)

(72) Inventor: Keshav MALSHE, Orlando, FL (US)

(21) Appl. No.: 15/103,072

(22) PCT Filed: Dec. 9, 2014
(86) PCT No.: PCT/US14/69312

§ 371 Date: Jun. 9, 2016

Related U.S. Application Data
(60) Provisional application No. 61/913,561, filed on Dec. 9, 2013.

(51) Int. Cl. A61K 38/27; A61K 45/06; A61K 9/00; A61K 31/4166; A61K 31/375

(52) U.S. Cl. CPC A61K 38/27; A61K 31/4166; A61K 31/375; A61K 9/0014; A61K 45/06

(57) ABSTRACT

The present invention is directed to a wound composition comprising an effective amount of phenytoin and/or an effective amount of vitamin C, and an effective amount of a cell growth stimulating compound. The present invention is further directed to a method of treating a wound in a patient comprising administering an effective amount of phenytoin and an effective amount of vitamin C to the wound.

### FIELD OF THE INVENTION

The present invention relates to compositions comprising a mixture of phenytoin, vitamin C, and/or growth hormone, or other conjunctive compounds, for use in the treatment of wounds, e.g., diabetic ulcers, and to methods for treating such wounds with the compositions.

### BACKGROUND OF THE INVENTION

Generally, when the skin of an individual is torn, cut or punctured (wounded), the body naturally reacts to regenerate dermal and epidermal tissue to close the wound. The wound regeneration process typically includes a set of complex biochemical events that take place in a closely orchestrated cascade to repair the damage. These events overlap in time, but may be categorized into different phases, namely the inflammatory, proliferative, and remodeling phases.

In the inflammatory phase, bacteria and debris are phagocytized and removed, and factors are released that cause the migration and division of cells involved in the proliferative phase. In the proliferative phase, the principal steps include angiogenesis, fibroplasias, granulation tissue formation, epithelialization, and wound contraction. Angiogeneis involves the development of new capillary blood vessels for the wound area to provide oxygen and nutrients to the healing tissue. In fibroplasia and granulation tissue formation, fibroblasts grow and form a new, provisional extracellular matrix (ECM) by excreting collagen and fibronectin. In epithelialization, epithelial cells crawl across the wound bed to cover the bed. In contraction, the wound is made smaller by the action of myofibroblasts, which establish a grip on the wound edges and contract themselves using a mechanism similar to that in smooth muscle cells. When the cells' roles are close to complete, unneeded cells undergo apoptosis.

It is known that a number of disease states hinder the normal wound healing process. For example, individuals with diabetes often experience problems with what are termed “diabetic foot ulcers.” Diabetic foot ulcers are sores or wounds, typically, on the feet that typically occur in individuals having diabetes. Oftentimes, these diabetic ulcers occur as a direct or indirect result of nerve damage in the feet of the individual as the prolonged high blood sugar levels associated with diabetes is linked with damage to the nerves in the feet. Such nerve damage in the feet, referred to as peripheral neuropathy, can cause loss of sensation as well as cause deformities of the feet. Due to the loss of sensation, individuals with peripheral neuropathy may hurt their feet by repetitive minor trauma (e.g., by prolonged walking) or a single major trauma (e.g., by scraping skin, stepping on objects, immersing feet in hot water, cutting toenails inappropriately, or wearing ill-fitting shoes), but nevertheless may not notice such injuries. A further complication of diabetes is a reduction in blood flow to the feet due to the arterial blockage or other causes, thereby severely inhibiting the body's ability to adequately provide complete the proliferative stage of wound regeneration/healing described above. As a result, once the skin of the foot is torn, cut, or punctured, the wound healing process (e.g., the proliferative phase) may be inordinately slow in repairing the wound. Further, once a serious wound develops, the risk of infection is high as the individual's body is simply unable to heal the wound. Even further, once an infection starts, the infection may be very difficult to reverse, and amputation of the affected limb is common

A number of treatments have been proposed to speed wound healing in patients having diabetic ulcers. These treatments include the use of skin grafts or “tissue equivalents.” Tissue equivalents involve the isolation of replacement skin cells that are expanded and seeded onto or into a supporting structure, such as a three-dimensional bioresorbable matrix, or within a gel-based scaffold. Both skin grafts and tissue equivalents are notably complex and, especially in the case of reduced blood flow to the patient's legs or feet, are often unsuccessful. Other treatments involve the direct application or injection at the wound site of particular pharmaceutical agent(s). Phenytoin, for example, is a drug that has been used for decades in the treatment of convulsive disorders. Recently, however, phenytoin has been investigated for its wound healing properties in the treatment of diabetic ulcers. See Bhatia, A., Prakash, S., Topical Phenytoin for Wound Healing. Dermatology Online Journal 2004; 10(1):5; and Mutyhukumarasamy M G, Sivakumar G, Manoharan G. Topical phenyotin in diabetic foot ulcers. Diabetes Care 1991; 14:909-11. Mutyhukumarasamy et al. allegedly found an improvement in the mean healing time for patients with diabetic ulcers in patients treated with phenytoin (21 days) vs. a control group (45 days). Nevertheless, the use of phenytoin for the control and healing of wounds, including diabetic ulcers, has not become a standard treatment, primarily because the incidence of amputation remains high and the degree of wound healing remains insufficient. Accordingly, there remains a need for an improved composition and method of treatment for diabetic ulcers.

### DETAILED DESCRIPTION OF THE INVENTION

The present inventors have surprisingly found that the combination of phenytoin and vitamin C provides improved wound healing properties to a patient over the use of either phenytoin or vitamin C alone. Thus, the present invention provides a potent wound healing and potentially limb-saving composition utilizing readily available pharmaceutical products.

In accordance with one aspect of the present invention, there is provided a wound composition comprising an effective amount of phenytoin and an effective amount of vitamin C. The phenytoin and vitamin C may be provided as a single composition or may be applied independent of one another.

In accordance with another aspect of the present invention, there is provided a pharmaceutical composition comprising an effective amount of phenytoin and an effective amount of vitamin C and at least one pharmaceutical acceptable excipient.

In accordance with yet another aspect of the present invention, there is provided an article of manufacture comprising packaging material, an effective amount of phenytoin, and an effective amount vitamin C, wherein the packaging material comprises a label or package insert indicating that the effective amount of phenytoin and the effective amount of vitamin C can be administered to a patient for accelerating a healing process of a wound.

In accordance with yet another aspect of the present invention, there is provided a method of treating a wound in a patient comprising administering topically an effective amount of phenytoin and an effective amount of vitamin C to the wound.

In accordance with yet another aspect of the present invention, there is provided a method of treating a wound in a patient comprising administering parenterally an effective amount of phenytoin and an effective amount of vitamin C to the wound.

In accordance with yet another aspect of the present invention, there is provided a method of accelerating the healing of a wound in a patient comprising administering an effective amount of phenytoin and an effective amount of vitamin C to the wound.

In accordance with yet another aspect of the present invention, there is provided a method for treating a diabetic ulcer comprising administering to a patient in need thereof a wound composition comprising an effective amount of phenytoin and an effective amount of vitamin C.

### Definitions

As used herein, the use of the word “a” or “an” when used in conjunction with the term “comprising” in the claims and/or the specification may mean “one,” but it is also consistent with the meaning of “one or more,” “at least one,” and “one or more than one.”

The term “wound” as used herein refers to any break in the epithelium resulting from a cut, abrasion, adhesion, surgical incision, thermal, chemical, or friction burn, ulcer, or the like, as a result of an accident, incident, surgical procedure, or the like. Wound can be further defined as acute and/or chronic. Compositions of the present invention have been found to be particularly useful in the treatment of diabetic ulcers, which are a type of wound as defined herein.

The term “pharmaceutically acceptable carrier” as used herein includes any and all solvents, dispersion media, coatings, antibacterial and antifungal agents, isotonic and absorption delaying agents, and the like. The use of such media and agents for pharmaceutically active substances is well known in the art.

The term “pharmaceutical composition” as used herein refers to a wound composition as described herein that this dispersed in a pharmaceutically acceptable carrier.

The term “patient” as used herein refers to any mammalian patient to which a wound composition is administered according to the methods described herein. In a specific embodiment, the compositions and methods of the present invention are employed to treat a human patient.

The term “effective amount” as used herein refers to an amount effective, at dosages and for periods of time necessary to achieve a desired result. The desired result may be an improvement in the wound healing process (e.g., by reducing a surface area of the wound), remediation of the symptoms of the wound (e.g., pain, infection, and the like), shortening of the duration of any stage in the wound healing process, stabilization of the state of wound (e.g., infection), prevention or slowing of the development of wound progression, prevention of, delay or slowing of wound progression, delay or slowing of wound onset, amelioration or palliation of the wound state, and remission (whether partial or total), whether detectable or undetectable.

The term “topical administration” as used herein includes the contact of the wound compositions of the present invention with tissue on or about the wound area.

The term “parenteral administration” as used herein includes any form of administration in which a composition is delivered or absorbed into the patient without involving absorption via the intestines. Exemplary parenteral administrations that are used in the present invention include, but are not limited to intradermal or subcutaneous administration.

The term “treating” and “treatment” as used herein refers to an approach for obtaining beneficial or desired results, including clinical results. Beneficial or desired clinical results can include, but are not limited to, an improvement in the wound healing process (e.g., by reducing a surface area of the wound), remediation of the symptoms of the wound (e.g., pain, infection, and the like), shortening of the duration of any stage in the wound healing process or the overall wound healing process, stabilization of infection (if any), and remission (whether partial or total), whether detectable or undetectable.

As used herein, the term “phenytoin” includes phenytoin, any pharmaceutically acceptable salt thereof, and/or any derivative thereof. Phenytoin was first synthesized by German chemist Heinrich Biltz in 1908. Phenytoin has the chemical structure, C₁₅H₁₂N₂O₂, and is also known as diphenylhydantoin or 5.5-diphenyl-2,4-imidazolidinedione. Phenytoin is commercially available worldwide as phenytoin or in the form of a pharmaceutically acceptable salt, e.g., phenytoin sodium, under the names Phenytek®, Dilantin®, Dilantin® Kapseals®, Dilantin® Infatabs®, Epotoin, Diphenin, Difnenin, Dipheninum, and/or Phydum™, for example. Exemplary phenytoin derivatives are set forth in U.S. Pat. No. 5,306,617 and in EP 0471338 B1, the entirety of each of which are hereby incorporated by reference.

As used herein, “Vitamin C” refers to ascorbic acid, the reduced form of ascorbic acid (ascorbate), mineral ascorbates (such as sodium, potassium, calcium, zinc, molybdenum, chromium and manganese ascorbates), ascorbyl palmitate, D-isoascorbic acid, and any isomers and/or esters of any of the aforementioned compounds. Derivatives and/or analogues of Vitamin C are included as well. U.S. Pat. Nos. 5,801,192 and 6,744,114 are cited to and incorporated herein in its entirety for teaching on vitamin C derivatives and analogues that may be used as the vitamin C component. While not wishing to be bound by theory, it is believed that Vitamin C is therapeutically effective for the production and maintenance of collagen, as well as for the metabolism of folic acid, tyrosine and tryptophan. Vitamin C is also believed to enhance the body's immune response, thereby protecting against infection, and aiding in the production of thyroxine. Of importance, Vitamin C also is further believed to have antioxidant properties. Vitamin C may be derived from any natural source, such as Vitamin C extracted from rose hips, acerola cherries, peppers, citrus fruits, corm syrup, or sago palm, or may be produced synthetically. Vitamin C is also readily commercially available from numerous commercial sources. When added to the wound compositions of the present invention, Vitamin C synergistically improves the therapeutic effectiveness of the phenytoin.

The term “consisting essentially of” or “consists essentially of” in relation to describing or claiming a composition relates to the inclusion of phenytoin, cell growth stimulating compound, and/or vitamin C, and excludes other active compounds. However, the term “consisting essentially of” as used herein may include cosmetic ingredients, pharmaceutically acceptable topical carriers, or other excipients.

The term “cell growth stimulating compound” is used throughout the specification and claims to describe those compounds which are added to the formulations according to the present invention for their known benefits in stimulating the growth and elaboration of cells. Cell growth stimulating compounds for use in the present invention include anabolic protein growth hormones, such as human growth hormone (GH), and related animal growth hormones, other non-steroidal anabolic hormones, for example, thyroxin (T₄), tri-iodothyronine (T₃) and insulin, among others, and growth factors, including for example, epithelial growth factor (EGF), fibroblast growth factor (FGF), transforming growth factor (TGF) and insulin-like growth factor (IGF). In the formulations according to the present invention, one or more cell growth stimulating compound is included in an amount effective for stimulating the growth of cells which surround, have been injured by or are responsible for healing a wound. Cell growth stimulating compounds for use in the present invention may include naturally isolated or synthetically produced versions of the above-mentioned compounds or their equivalents and include, where relevant, compounds produced by genetic engineering processes and techniques. Cell growth stimulating compounds also may relate to The therapeutic agent may include, but is not limited to, members of the fibroblast growth factor family, including acidic and basic fibroblast growth factor (FGF-1 and FGF-2) and FGF-4, members of the platelet-derived growth factor (PDGF) family, including PDGF-AB, PDGF-BB and PDGF-AA; EGFs; the TGF-β superfamily, including TGF-β1, 2 or 3; osteoid-inducing factor (OIF); angiogenin(s); endothelins; hepatocyte growth factor or keratinocyte growth factor; members of the bone morphogenetic proteins (BMP's) BMP-1, BMP-3, BMP-2; OP-1, BMP-2A, BMP-2B, or BMP-7; HBGF-1 or HBGF-2; growth differentiation factors (GDF's); members of the hedgehog family of proteins, including indian, sonic and desert hedgehog; ADMP-1; other members of the interleukin (IL) family; or members of the colony-stimulating factor (CSF) family, including CSF-1, G-CSF, and GM-CSF, or isoforms thereof; or VEGF, NELL-1 (neural epidermal growth factor-like 1), CD-RAP (cartilage-derived retinoic acid-sensitive protein) or combinations thereof.

In some embodiments, the composition comprises osteogenic proteins as cell growth stimulating compounds. Exemplary osteogenic proteins include, but are not limited to, OP-1, OP-2, OP-3, BMP-2, BMP-3, BMP-3b, BMP-4, BMP-5, BMP-6, BMP-9, BMP-10, BMP-11, BMP-12, BMP-13, BMP-14, BMP-15, GDF-1, GDF-2, GDF-3, GDF-5, GDF-6, GDF-7, GDF-8, GDF-9, GDF-10, GDF-11, GDF-12, CDMP-1, CDMP-2, CDMP-3, DPP, Vg-1, Vgr-1, 60A protein, NODAL, UNIVIN, SCREW, ADMP, NEURAL, and TGF-beta. As used herein, the terms “morphogen, ” “bone morphogen, ” “BMP, ” “osteogenic protein” and “osteogenic factor” embrace the class of proteins typified by human osteogenic protein 1 (hOP-1).

Exemplary growth factors include, but are not limited to, members of the transforming growth factor beta family, including bone morphogenetic protein 2 (BMP-2); bone morphogenetic protein 4 (BMP-4); and transforming growth factors beta-1, beta-2, and beta-3 (potent keratinocyte growth factors). Other useful members of the transforming growth factor beta family include BMP-3, BMP-5, BMP-6, BMP-9, DPP, Vgl, Vgr, 60A protein, GDF-1, GDF-3, GDF-5, GDF-6, GDF-7, CDMP-1, CDMP-2, CDMP-3, BMP-10, BMP-11, BMP-13, BMP-15, Univin, Nodal, Screw, ADMP, Neural, and amino acid sequence variants thereof. Other growth factors include epidermal growth factor (EGF), which induces proliferation of both mesodermal and ectodermal cells, particularly keratinocytes and fibroblasts; platelet-derived growth factor (PDGF), which exerts proliferative effects on mesenchymal cells; fibroblast growth factor (FGF), both acidic and basic; and insulin-like growth factor 1 (IGF-1) or 2 (IGF-2), which mediate the response to growth hormone, particularly in bone growth. Further growth factors include osteogenic proteins. A particularly preferred osteogenic protein is OP-1, also known as bone morphogenetic protein 7 (BMP-7). OP-1 is a member of the transforming growth factor beta gene superfamily.

While not wishing to be bound by theory, it is believed that when applied to a wound, such as a diabetic ulcer, the phenytoin of the wound compositions of the present invention may assist wound healing by one or more of processes, such as by stimulating fibroblast proliferation, enhancing the formation of granulation tissue, decreasing collagenase activity, promoting deposition of collagen and other connective components, decreasing bacterial contamination, and decreasing wound exudate. See Bhatia, A., Prakash, S., Topical Phenytoin for Wound Healing. Dermatology Online Journal 2004; 10(1):5. Mutyhukumarasamy M G, Sivakumar G, Manoharan G. Topical phenyotin in diabetic foot ulcers. Diabetes Care 1991; 14:909-11.

The present inventors have surprisingly found, however, that when an amount of Vitamin C is added to a wound composition comprising phenytoin, the therapeutic effectiveness of the wound composition in treating wounds, e.g., diabetic ulcers, is markedly enhanced relative to phenytoin or vitamin C alone, and other compositions. Diabetic ulcers are notoriously difficult to control and treat due to decreased blood flow to the affected area, associated nerve and tissue damage (if present), age, condition of the patient, and other factors. Often, when the wound will not properly heal and infection reaches an unacceptable or dangerous state, the limb of the individual is amputated above the wound site. The wound compositions of the present have proven to be effective in promoting wound healing and reducing the likelihood of amputation of the affected limb.

In an embodiment, the wound compositions of the present invention may also comprise an additional component, or component that substitutes for vitamin C, that acts as an antioxidant and/or further aids in promoting or improving wound healing relative to a composition without the additional component. For example, in one embodiment, the wound composition may further comprise an effective amount of vitamin E. By “Vitamin E,” it is meant any one or combination of the eight forms of Vitamin E comprising the four tocopherols and the four tocotrienols, including any succinate, nicotinate and acetate salts derivatives thereof. Each of these forms of Vitamin E has a “d” form, which is the natural form, and a “dl” form, which is the synthetic form. In one embodiment, the composition of the present invention comprises d-α tocopherol, or a salt derivative thereof, as this is the most active form of Vitamin E. Alternatively, in another embodiment, an effective amount of Vitamin E is utilized in the wound composition in place of the vitamin C. Vitamin E is believed to have anti-inflammatory effect when applied topically, as well as antioxidant properties. Vitamin E may be extracted from such natural products, such as vegetable oils (olive, sunflower, and safflower oils), nuts, whole grains, and green leafy vegetables. Alternatively, Vitamin E is also readily commercially available from numerous commercial sources. In another embodiment, the composition includes foliate, or folic acid, and/or magnesium, in combination with phenytoin, with or without vitamin C and/or vitamin E.

The wound compositions of the present invention may be provided in any suitable form for administration to a wound of a patient. For example, the wound compositions of the present invention may be provided and/or manufactured in a formulation for topical administration and/or parenteral administration, such as a topical formulation, an injectable formulation, and/or any other suitable formulation. It is understood that the active ingredients may be provided individually (with or without instructions for future mixing) or together (mixed, non-mixed, or with instructions for future mixing) in the respective formulation. Further, the wound composition may be prepared by known methods for the preparation of pharmaceutically acceptable compositions suitable for administration to a patient, such that an effective quantity of the active ingredients is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985).

In one embodiment, the wound composition is provided in the form of a topical composition, such as a powder, ointment, lotion, cream, and/or gel. In a particular embodiment, the topical composition may be provided in the form of a powder. In another embodiment, the active compounds of the composition are mixed in a composition that utilizes honey as a carrier. The topical composition may comprise phenytoin in an amount from about 100 g to 1000 g, and in a particular embodiment about 300 g. Similarly, the topical composition may comprise Vitamin C in an amount of about 100 g to 1000 g, and in a particular embodiment about 500 g. The ratio of phenytoin to vitamin C may be from about 1:10 to 1:1 by weight. In a particular embodiment, the ratio of phenytoin to vitamin C may be from about 1:5 to about 1:1 by weight, e.g. 300 mg phenytoin to 500 mg vitamin C.

When a wound composition of the present invention is provided in the form of a topical composition, e.g., powder, ointment, lotion, cream, and/or gel, the wound composition may further include other cosmetic ingredients and pharmaceutically acceptable topical carriers which have substantially non-irritating, skin compatible components. Such suitable cosmetic and pharmaceutical agents include, but are not limited to, moisturizers, stabilizers, preservatives, antiseptics, lubricants, humectants, gelling agents, chelating agents, skin penetration enhancers, emollients, colorants, solvents, fatty bodies, thickening agents, emulsifiers, and/or any other excipient, which does not alter the therapeutic effect of the active ingredients, e.g., phenytoin and vitamin C.

When any one of the wound compositions described herein is provided in the form of an injectable solution, the injectable solution may comprise phenytoin in an amount from about 200 g to 400 g, and in a particular embodiment, about 300 mg. In addition, the injectable solution may comprise Vitamin C in an amount from about 100 g to 1000 g, and in particular embodiment, about 500 g. The ratio of phenytoin to vitamin C may be from about 1:10 to 1:1 by weight. In a particular embodiment, the ratio of phenytoin to vitamin C may be from about 1:5 to about 1:1 by weight, e.g. 300 mg phenytoin to 500 mg vitamin C. Optionally, the phenytoin and the vitamin C may be mixed with a suitable of 10 N saline to form the injectable solution.

In addition, when provided in the form of in injectable solution, the injectable solution may further include any suitable pharmaceutically acceptable additives. Suitable pharmaceutically acceptable additives include additives employed customarily in a injection formulation, such as a stabilizer (ascorbic acid, sodium pyrosulfite and the like), a surfactant (polysorbate 80, macrogol and the like), a solubilizing agent (glycerin, ethanol and the like), a buffering agent (phosphoric acid and its alkali metal salt, citric acid and its alkali metal salt and the like), an isotonizing agent (sodium chloride, potassium chloride and the like), a dispersing agent (hydroxypropylmethyl cellulose, dextrin), a pH modifier (hydrochloric acid, sodium hydroxide and the like), a preservative (ethyl p-oxybenzoate, benzoic acid and the like), a solubilizer (concentrated glycerin, meglumine and the like), a solubilizing aid (propylene glycol, sugar and the like), and/or a painkiller (glucose, benzyl alcohol and the like) as desired. Any of these agents or additives may be added in an amount employed customarily in a formulation for injection, and preferably do not alter the therapeutic effect of the active ingredients of the present invention, e.g., phenytoin and vitamin C.

In an embodiment, the present invention also provides an article of manufacture comprising packaging material and an effective amount of phenytoin and vitamin C for the treatment of a wound, e.g., a diabetic ulcer. The packaging material may comprise a label or package insert indicating that the effective amount of phenytoin and the effective amount of vitamin C can be administered to a patient, e.g. a human patient, for inducing or accelerating healing of the wound. The label or package insert may comprise user-readable and/or computer-readable information embodied on any suitable medium, such as a paper insert or a computer-readable disc.

When a wound composition as described herein is a topical formulation, the wound composition may be applied directly to the wound in an amount effective to substantially cover the wound with the wound composition. Also, when the wound composition is a topical composition, e.g., cream, ointment, or the like, it is generally desirable to apply the wound composition directly to the wound by any suitable sterile applicator or sterile method of application known in the art.

In addition, when a wound composition as described herein is provided as an injectable solution, the injectable solution may be injected directly into the wound of the patient, such as by intradermal or subcutaneous injection, or otherwise injected into an about the wound area of the patient using a sterile syringe or other suitable device.

When applying and/or injecting the wound composition onto and/or to the wound, it may be desirable to administer a suitable analgesic by any suitable method known in the art beforehand, e.g., benzyl alcohol, chlorobutanol, to reduce pain and/or further inflammation in the wound area.

One skilled in the art would readily appreciate that the administration duration and dosage of the wound composition may be determined or adjusted based on the age, body weight, general condition, sex, diet, the severity of the wound, and/or degree of inflammation and/or infection associated with the wound. Effective amounts of the wound composition can be given repeatedly, depending upon the effect of the initial treatment regimen. Administrations are typically given periodically, while monitoring any response. It will be recognized by a skilled person that lower or higher dosages or number of applications other than those indicated herein may be given, according to the administration schedules and routes selected.

In an embodiment, the present inventors have found the wound compositions of the present invention are particularly effective when administered at least once daily for a first week's time. Thereafter, the wound compositions may be applied to the wound at least once a week until such time as the wound is satisfactorily healed. In one embodiment, a wound composition is applied once a week (after the initial daily application) for a period of at least seven weeks. It is understood that prior to each application, the wound may be rinsed with a sterile solution, such as saline, and allowed to dry. Thereafter, a wound composition may be applied and the wound wrapped in a suitable dressing.

An exemplary fresh diabetic ulcer of an exemplary patient is shown in FIG. 1. Upon treatment with a wound composition of the present invention, new signs of skin formation are likely to be present within three weeks (wound healing by secondary intention), particularly if the composition if applied to the wound daily for at least the first 7-10 days. Within weeks (after the daily application), it is expected that the wound will begin to appear generally healthier, that granulation tissue will be deposited, and that wound will begin to close as is generally shown in FIG. 2. After 10-18 weeks of weekly treatment, it is expected that the wound will be 90-100% closed and healed, see FIGS. 3-4.

It is further understood that the wound compositions of the present invention as described herein may be employed alone in the treatment, or may be combined with a concomitant drug, which is administered to the patient independently of the wound composition or as part of the same formulation. Such concomitant drugs may be delivered simultaneously, before and/or after the administration of the wound composition. Suitable concomitant drugs may include, by way of example only, vasoldilators, vasoconstrictors, hypertensive agents, antibacterial agents, antibiotics, antifungal agents, non-steroidal antiinflammatory agents, steroidal agents, anesthetics, and/or diabetes agents.

Suitable vasodilators include, but are not limited to, manidipine, nicardipine, nilvadipine, nisoldipine, nitrendipine, benidipine, amlodipine, aranidipine, budralazine, cadralazine, ecarazine, hydralazine, todralazine, oxyphedrine, diltiazem, tolazoline, hexobendine, bamethan, clonidine, methyldopa, guanabenz, and the like.

Suitable vasoconstrictors include, but are not limited to, dopamine, dobutamine denopamine and the like.

Suitable hypertensive agents include, but are not limited to, dopamine, dobutamine, denopamine, digitoxin, digoxin, methyldigoxin, lanatoside C, G-strophantin, and the like.

Suitable antibacterial agents include, but are not limited to, sufonamides, such as sulfamethizole, sulfisoxazole, sulfamonomethoxin, sulfamethizole, salazosulfapyridine, silver sulfadiazine, and the like, and quinolones, such as nalidixic acid, pipemidic acid trihydrate, enoxacin, norfloxacin, ofloxacin, tosufloxacin tosilate, ciprofloxacin hydrochloride, lomefloxacin hydrochloride, sparfloxacin, fleroxacin, and the like.

Suitable antibiotics include, but are not limited to, tetracyclin hydrochloride, ampicillin, piperacillin, gentamycin, dibekacin, kanendomycin, lividomycin, tobramycin, amikacin, fradiomycin, sisomicin, tetracyclin, oxytetracyclin, rolitetracyclin, doxycyclin, ampicillin, piperacillin, ticarcillin, cefalotin, cefapirin, cefaloridine, cefaclor, cefalexin, cefroxadine, cefadroxil, cefamandole, cefotoam, cefroxime, cefotiam, cefotiam hexetil, cefuroxime axetil, cefdinir, cefditoren pivoxil, ceftazidime, cefpiramide, cefsulodin, cefinenoxime, cefpodoxime proxetil, cefpirome, cefozopran, cefepime, cefsulodin, cefinenoxime, cefinetazole, cefminox, cefoxitin, cefbuperazone, latamoxef, flomoxef, cefazolin, cefotaxime, cefoperazon, ceftizoxime, moxalactam, thienamycin, sulfazecin, azthreonam and their salts, griseofulvin, lankacidin [J. Antibiotics, 38, 877-885 (1985)], and the like.

Suitable antifungal agents include, but are not limited to, polyene-based antibiotics (e.g., amphotericin B, nystatin, trichomycin); griseofulvin, pyrrolnitrin, and the like; cytosine metabilism antagonists (e.g., flucytosine); imidazole derivatives (e.g., econazole, clotrimazole, miconazole nitrate, bifonazole, croconazole); triazole derivatives (e.g., fluconazole, itraconazole, azole-based compounds, e.g., [2-[1R, 2R)-2-(2,4-difluorophenyl)-2-hydroxy-1-methyl-3-(1H-1,2,4-triazol-1-yl)propyl]-4-[4-(2,2,3,3-tetrafluoropropoxylphenyl-3-(2H,4H)-1,2,4-triazolone); thiocarbamic acid derivatives (e.g., trinaphthol); echinocandin-based derivatives (e.g., caspofamgine, FK-463, V-Echinocadin), and the like.

Suitable non-steroidal antiinflammatory agents include, but are not limited to, acetaminophen, fenasetin, ethenzamide, sulpyrine, antipyrine, migrenin, aspirin, mefenamic acid, flufenamic acid, diclofenac sodium, loxoprofen sodium, phenylbutazone, indomethacin, ibuprofen, ketoprofen, naproxen, oxaprozin, flurbiprofen, fenbufen, pranoprofen, floctafenine, epirizol, tiaramide hydrochloride, zaltoprofen, gabexate mesilate, camostat mesilate, ulinastatin, colchicine, probenecid, sulfinpyrazone, benzbromarone, allopurinol, sodium gold thiomalate, sodium hyaluronate, sodium salicylate, morphine hydrochloride, salicylic acid, atropine, scopolamine, morphine, pethidine, levorphanol, ketoprofen, naproxen, oxymorphone, and their salts.

Suitable steroidal agents include, but are not limited to, dexamethasone, hexestrol, methimazole, betamethasone, triamcinolone, triamcinolone acetonide, fluorocinonide, fluorocinolone acetonide, prednisolone, methylprednisolone, cortisone acetate, hydrocortisone, fluorometholone, beclometasone dipropionate, estriol, and the like.

Suitable anesthetics include, but are not limited to, cocaine hydrochloride, procaine hydrochlodie, lidocaine, dibucaine hydrochloride, tetracaine hydrochloride, mepivacaine hydrochloride, bupivacaine hydrochloride, oxybuprocaine hydrochloride, ethyl aminobenzoate, oxethazaine, and the like, or other systemic, inhalation, or intravenous anesthetics.

Suitable diabetes agents include, but are not limited to, actos, lodiglitazon, kinedak, penfill, humalin, euglucon, glimicron, daonil, novolin, monotard, insulins, glucobay, dimelin, rastinon, bacilcon, deamelin S, Iszilins]; hypothyroidism treating agent [dried thyroid gland (thyreoid), levothyroxine sodium (thyradin S), liothyronidin sodium (thyronine, thyromin), and the like.

The ratio between a compound of the present invention and a concomitant drug in a concomitant formulation may be selected appropriately on the basis of the target, route, and disease/condition of the patient and may be readily determined by one skilled in the art. In a particular embodiment, the present invention includes a method of treating a wound comprising administering to a patient in need thereof a wound composition comprising an effective amount of phenytoin and an effective amount of vitamin C along with an effective amount of an antibiotic to aid in combating wound infection.

The following examples are not understood to be limiting, but are useful in illustrating the effectiveness of a wound composition comprising phenytoin and vitamin C over a wound composition having phenytoin alone as an active ingredient.

The teachings of the references cited throughout the specification are incorporated herein in their entirety by this reference to the extent they are not inconsistent with the teachings herein. It should be understood that the examples and the embodiments described herein are for illustrative purposes only and that various modifications or changes in light thereof will be suggested to persons skilled in the art and are to be included within the spirit and purview of this application.

The invention claimed is:

1. A wound composition comprising an effective amount of phenytoin, and/or an effective amount of vitamin C, and an effective amount of a cell growth stimulating compound.

2. The wound composition of claim 1, wherein the cell growth stimulating compound is human growth hormone.

3. The wound composition of claim 1, wherein the wound composition consists essentially of phenytoin, vitamin C and human growth hormone.

4. The wound composition of claim 1, wherein the wound composition comprises a powder.

5. The wound composition of claim 1, wherein the wound composition comprises at least one of an ointment, lotion, cream, and/or gel.

6. An article of manufacture comprising packaging material and an effective amount of phenytoin and/or an effective amount of vitamin C, and an effective amount of human growth hormone, wherein said packaging material comprises a label or package insert indicating that said effective amount of phenytoin, said effective amounts can be administered to a patient for inducing or accelerating a healing process of a diabetic ulcer.

7. A pharmaceutical composition comprising an effective amount of phenytoin and/or an effective amount of vitamin C, and an effective amount of a cell growth stimulating compound, and at least one pharmaceutical acceptable excipient.

8. The composition of claim 7, wherein said cell growth stimulating compound is human growth hormone.

9. A method of treating a patient having a wound, the method comprising administering via topical administration an effective amount of the composition of claim 1 to or in the vicinity of the wound.

10. A method for treating a diabetic ulcer comprising administering to a patient in need thereof a wound composition comprising an effective amount of phenytoin and/or an effective amount of vitamin C, and an effective amount of cell growth stimulating compound,

11. The method of claim 10, wherein said cell growth stimulating compound is human growth hormone.

12. The method of claim 10, wherein the wherein the wound composition consists essentially of phenytoin, vitamin C and human growth hormone.

13. The method of claim 10, wherein the wound composition is administered via topical administration.

14. The method of claim 10, wherein the wound composition is administered via parenteral administration.

15. The method of claim 10, wherein the wound composition is administered to the patient at least once daily for at least seven days, and thereafter at least once weekly for at least eight weeks.

16. The wound composition of claim 1, wherein said composition further comprises vitamin E, folic acid, and/or magnesium.

17. The wound composition of claim 16, wherein the wound composition comprises a powder.

18. The wound composition of claim 16, wherein the wound composition comprises at least one of an ointment, lotion, cream, and/or gel.

19. The wound composition of claim 1, further comprising an antifungal agent.

20. The wound composition of claim 1, further comprising an antibacterial agent.
